(19) United States
(12) Patent Application Publication
(10) Pub. No.: US 2010/0125134 A1
(43) Pub. Date: May 20, 2010

(54) METHOD OF SEPARATING GENOMIC DNA AND PLASMID DNA FROM EACH OTHER AND KIT THEREFOR

(75) Inventors: Hee-kyun LIM, Hwaseong-si (KR); Kyu-youn HWANG, Yongin-si (KR); Joon-ho Kim, Seongnam-si (KR)

Correspondence Address: CANTOR COLBURN, LLP, 20 Church Street, 22nd Floor, Hartford, CT, 06103, US

(73) Assignee: Samsung Electronics, Co., Ltd., Gyeonggi-do (KR)

(21) Appl. No.: 12/611,201

(22) Filed: Nov. 3, 2009

(30) Foreign Application Priority Data
Nov. 19, 2008 (KR) 10-2008-0115326

(51) Int. Cl. C07H 21/04

(52) U.S. Cl. 536/23.1

(57) ABSTRACT

Provided are a method of separating genome DNA and plasmid DNA from each other from a sample using a binding buffer containing a high concentration kosmotropic salt and chaotropic salt, and a kit therefor.

### CROSS-REFERENCE TO RELATED APPLICATIONS

This application claims priority to Korean Patent Application No. 10-2008-0115326, filed on Nov. 19, 2008, and all the benefits accruing therefrom under 35 U.S.C. §119, the contents of which in their entirety are herein incorporated by reference.

### BACKGROUND

1. Field

One or more embodiments relate to a method of separating genome DNA and plasmid DNA from each other and a kit therefor.

2. Description of the Related Art

Separation of plasmid DNA from samples, particularly, biological samples, needs to be performed in advance for molecular biological assays such as polymerase chain reactions (“PCRs”), sequence analyses, cloning, and transformation. Recently, plasmid DNA (“pDNA”) has been widely applied to vaccines, gene therapies, etc. Accordingly, the separation of plasmid DNA is considered to be important not only in research of molecular biology and biological engineering fields, but also in diagnoses, treatments, and medical applications.

Conventionally, pDNA is separated by lysing cells in an alkali condition, adding acid to the cell lysates to prepare weak acidic cell lysates, adding ethanol thereto, and centrifuging the solution at high speed to precipitate genome DNA (“gDNA”). In this regard, the high-speed centrifugation can be performed at a speed of about 13,000 rpm (about 17,900 g) to about 14,000 rpm (about 20,000 g). Even though a commercially available kit of a QIAgen MiniPrep™ DNA (QIAgen) has been used, high-speed centrifugation is also used.

Microfluidic devices have been widely developed as devices used to analyze biological samples with high capacity and high efficiency. Microfluidic devices need to include motors suitable for precisely controlling components such as valves. For example, a servomotor may be used to precisely control mechanical position or other parameters. However, motors used to precisely control mechanical position do not provide sufficient centrifugal force to obtain high-speed centrifugation.

Thus, there is a need to develop alternative methods of efficiently separating gDNA and pDNA from samples. In particular, there is a need to develop methods of separating gDNA and pDNA from samples without using high-speed centrifugation.

### SUMMARY

One or more embodiments include a method of efficiently separating gDNA and pDNA from a sample using a buffer containing a high concentration kosmotropic salt and chaotropic salt.

One or more embodiments include a method of efficiently separating gDNA and pDNA from a sample using a buffer containing a high concentration kosmotropic salt and chaotropic salt and a solid support material capable of binding to DNA.

One or more embodiments include a kit for efficiently separating gDNA and pDNA from a sample comprising a binding buffer containing a high concentration kosmotropic salt and chaotropic salt, and a solid support material capable of binding to DNA.

Additional aspects will be set forth in part in the description which follows and, in part, will be apparent from the description, or may be learned by practice of the presented embodiments.

To achieve the above and/or other aspects, one or more embodiments may include a method of separating genomic DNA and plasmid DNA from each other, the method including: mixing a sample including genome DNA and plasmid DNA with a solution including a kosmotropic salt and a chaotropic salt to prepare a mixture having a pH ranging from about 3 to about 5; and separating the genome DNA and plasmid DNA from the mixture.

To achieve the above and/or other aspects, one or more embodiments may include an analytical material for separating genome DNA and plasmid DNA from each other in a sample including a kosmotropic salt, a chaotropic salt, and a solid support material that binds to DNA at a pH of about 3 to about 5. To achieve the above and/or other aspects, one or more embodiments may include a method of separating genomic DNA and plasmid DNA from each other, the method comprising mixing a sample comprising genome DNA and plasmid DNA with a solution comprising a kosmotropic salt and a chaotropic salt to prepare a mixture having a pH ranging from about 3 to about 5; contacting the mixture with a solid support material capable of binding to genome DNA to form a genome DNA-solid support material complex; separating the genome DNA-solid support material complex from the mixture; and separating the plasmid DNA from the remaining mixture from which the genome DNA-solid support material complex is separated, wherein the separating the plasmid DNA from the remaining mixture is performed by contacting the remaining mixture with a metal oxide, separating the metal oxide from the remaining mixture, and eluting plasmid DNA from the separated metal oxide.

### BRIEF DESCRIPTION OF THE DRAWINGS

These and/or other aspects will become apparent and more readily appreciated from the following description of the embodiments, taken in conjunction with the accompanying drawings of which:

FIG. 1 is an electrophoretogram illustrating DNA separated from controls 1 and 2 according to Example 1;

FIG. 2 is an electrophoretogram illustrating gDNA and pDNA separated using a binding buffer containing a high concentration kosmotropic salt and chaotropic salt;

FIG. 3 is an electrophoretogram illustrating DNA separated from controls 1 and 2 according to Example 2;

FIG. 4 is an electrophoretogram illustrating DNA separated using a high concentration kosmotropic salt and chaotropic salt, and a material with a hydrophobic surface according to Example 3;

FIG. 5 is an electrophoretogram illustrating DNA separated from controls 1 and 2 according to Example 3;

FIG. 6 is an electrophoretogram illustrating DNA separated using a high concentration kosmotropic salt and chaotropic salt, and a CST material according to Example 4; and

FIG. 7 is an electrophoretogram illustrating the influence of types of kosmotropic salts on the separation of gDNA and pDNA.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the invention.

Spatially relative terms, such as “under” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “under” relative to other elements or features would then be oriented “above” relative to the other elements or features. Thus, the exemplary term “under” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

Hereinafter, the invention will be described in detail with reference to the accompanying drawings.

One or more embodiments include a method of separating genomic DNA (gDNA) and plasmid DNA (pDNA) from each other, the method including: mixing a sample including gDNA and pDNA with a solution including a kosmotropic salt and a chaotropic salt to prepare a mixture having a pH ranging from about 3 to about 5; and separating gDNA and pDNA from the mixture.

According to another embodiment, a method of separating genomic DNA and plasmid DNA from each other, the method comprising mixing a sample comprising genome DNA and plasmid DNA with a solution comprising a kosmotropic salt and a chaotropic salt to prepare a mixture having a pH ranging from about 3 to about 5; contacting the mixture with a solid support material capable of binding to genome DNA to form a genome DNA-solid support material complex; separating the genome DNA-solid support material complex from the mixture; and separating the plasmid DNA from the remaining mixture from which the genome DNA-solid support material complex is separated, wherein the separating the plasmid DNA from the remaining mixture is performed by contacting the remaining mixture with a metal oxide, separating the metal oxide from the remaining mixture, and eluting plasmid DNA from the separated metal oxide.

According to the method, a sample including gDNA and pDNA is mixed with a solution including a kosmotropic salt and a chaotropic salt to prepare a mixture having a pH ranging from about 3 to about 5.

As used herein, the term “genome DNA” refers to DNA forming all or part of genomes of prokaryotic cells or eukaryotic cells. As used herein, the term “plasmid DNA” refers to an extra-chromosomal, independently replicating, circular DNA. The gDNA may be a genome of bacteria, and the pDNA may be plasmid derived from bacteria.

The sample may be any sample including gDNA and pDNA. For example, the sample may be a lysed biological sample. The lysed biological sample may be prokaryotic cell lysates, eukaryotic cell lysates, or a mixture thereof. For example, the lysed biological sample may be one selected from a group consisting of bacteria cell lysates, mold cell lysates, plant cell lysates, and animal cell lysates.

The lysed biological sample may be prepared using known methods, for example, ultrasonic treatment, thermal treatment, pressure treatment, enzyme treatment, and chemical treatment (e.g., alkali treatment). For example, the sample may be cell lysates obtained by treating lysed bacteria cells at a pH ranging from about 12 to about 13.

The “kosmotropic salt” and “chaotropic salt” used herein are materials that change secondary, tertiary, and/or quaternary structures of protein and/or nucleic acid but do not change a primary structure of the protein and/or nucleic acid. Solutes are defined as kosmotropic if they contribute to the stability and structure of water-water interactions. Kosmotropes cause water molecules to favorably interact, which also stabilizes intermolecular interactions in macromolecules. Thus, the kosmotropic salt stabilizes a subject material. In contrast, a chaotropic agent, is a substance which disrupts the three dimensional structure in macromolecules such as proteins, DNA, or RNA and denatures them. Thus, the chaotropic salt destabilizes a subject material. The kosmotropic salt and the chaotropic salt are related to the Hofmeister series according to the degree of influencing solubility of protein in a solution. According to the Hofmeister series, ions are classified into anionic series and cationic series according to their ability to change water structure. The anionic series are as follows:

SO₄²⁻<HPO₄²⁻<OH⁻<F⁻<HCOO⁻<CH₃COO⁻<Cl⁻<Br⁻<NO₃⁻<I⁻<SCN⁻<ClO₄⁻.

The cationic series are as follows:

NH₄⁺, Rb⁺, K⁺, Na⁺, Cs⁺, Li⁺, Ca²⁺, Mg²⁺, and Ba²⁺.

The kosmotropic salt may consist of an anion of the Hofmeister series that stabilizes water structure and its counter cation. The counter cation may be a cationic series of the Hofmeister series. The kosmotropic salt may be a salt consisting of an anion selected from a group consisting of sulfate (SO₄²⁻), phosphate (HPO₄²⁻), hydroxide (OH—), fluoride (F—), formate (HCOO—), and acetate (CH₃COO—), and its cation, but is not limited thereto. The kosmotropic salt induces crystallization of protein, functions as a salting-out ion for hydrophobic particles, and forms a water structure according to the Hofmeister series. The kosmotropic salt may include at least one anion selected from a group consisting of acetate, phosphate, sulfate, and citrate. For example, the kosmotropic salt may be potassium acetate or sodium acetate. The concentration of the kosmotropic salt may be in a range of about 0.3 to about 3 M.

The chaotropic salt may consist of an anion of the Hofmeister series that destabilizes water structure, and its counter cation. The counter cation may be a cationic series of the Hofmeister series. The chaotropic salt may consist of an anion selected from a group consisting of Cl⁻, Br⁻, NO₃⁻, I⁻, SCN⁻, and ClO₄⁻, and its counter cation. The chaotropic salt may be guanidine hydrochloride, guanidine isocyanate, sodium iodide, or guanidine thiocyanate. The concentration of the chaotropic salt may be in a range of about 3 to about 6 M.

However, the kosmotropic salt and the chaotropic salt are not limited to the anions and their counter cations of the Hofmeister series. In this regard, any material stabilizing or destabilizing the structure of protein and/or a nucleic acid may be used.

According to the method of the present embodiment, a molar concentration ratio of the kosmotropic salt to the chaotropic salt may be in a range of about 1:14 to about 1:2. The pH of the solution including the kosmotropic salt and the chaotropic salt may be in a range of about 3 to about 5.

For example, in the solution including the kosmotropic salt and the chaotropic salt, the concentration of the kosmotropic salt may be in a range of about 0.3 to 3 M, the concentration of the chaotropic salt may be in a range of about 3 to about 6 M, and a molar concentration ratio of the kosmotropic salt to the chaotropic salt may be in a range of about 1:14 to about 1:2

The method includes separating gDNA and pDNA from the mixture. The “separating gDNA and pDNA” used herein indicates selectively separating either the gDNA or the pDNA from the mixture. In this regard, the separating gDNA and pDNA includes not only purifying each of the separated gDNA and pDNA but also relatively increasing the concentration of each of the gDNA and pDNA. That is, the separating gDNA and pDNA may include purifying or concentrating either the gDNA or the pDNA. According to the concentration, the molar concentration of the gDNA and pDNA may be increased by 50%, 70%, or 90% from the initial concentration of either the gDNA or pDNA.

The separating operation may include contacting the mixture with a solid support and separating the solid support from the mixture.

The solid support may be any known solid support for binding to DNA at a pH of about 3 to about 5. For example, the solid support may be a bi-functional material, a hydrophobic material, or ChargeSwitch™ technology (“CST”) material.

The bi-functional material may be positively charged and capable of binding to nucleic acids at a first pH and negatively charged and capable of releasing the bound nucleic acids at a second pH. The bi-functional material may be immobilized to the solid support. The first pH may be in a range of about 2 to about 5, for example, about 3 to about 4.5 and about 3.5 to about 4.5. The second pH may be in a range of about 7 to about 12, for example, about 7 to about 11, about 8 to about 11, or about 8 to about 10.

The bi-functional material may be a compound represented by Formula 1 below.

Q-X-Q₁ Formula 1

In one embodiment, in Formula 1, Q or Q1 is a group represented by —X₁—R₁—Y₁, wherein X₁ is —NR₂—, wherein R₂ is selected from the group consisting of hydrogen and a substituted or unsubstituted C₁-C₁₀ alkyl group, O—, or —S—; wherein R₁ is selected from a group consisting of a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group; and wherein Y₁ is a group represented by

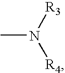

wherein R₃ and R₄ are each independently selected from a group consisting of hydrogen, a substituted or unsubstituted C₁-C₁₀ alkyl group, a substituted or unsubstituted C₃-C₁₀ cycloalkyl group, a substituted or unsubstituted C2-C10 alkenyl group, and a substituted or unsubstituted C₂-C₁₀ alkynyl group, or R₃ and R₄ are bound to each other to form an alicyclic or aromatic ring, or Y₁ is a group that is represented by

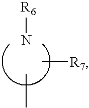

and has a 4- to 8-membered alicyclic ring or a 6- to 8-membered aromatic ring, wherein R₆ and R₇ are each independently selected from a group consisting of hydrogen, halogen (with a proviso that R₆ is not halogen), a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group, and at least one selected from the group consisting of Q and Q₁ has a primary or secondary amino group,

X is a compound represented by

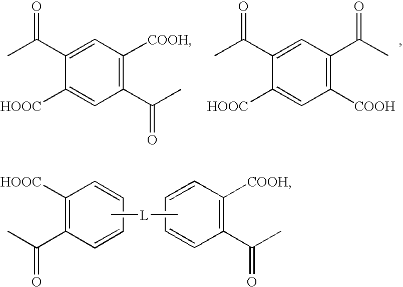

wherein L is a simple bond, —O—, —CO—, —S—, —SO₂—, —CH₂—, —C(CH₃)₂—, or —C(CF₃)₂—,

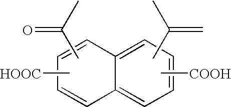

(wherein the carbonyl group and the carboxyl group are substituted at any carbon position except for a ring connection portion),

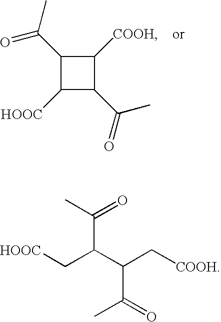

In one embodiment, in Formula 1, Q or Q₁ is a group represented by —X₁—R₁—Y₁, wherein X1 is —NR₂— where R₂ is selected from the group consisting of hydrogen and a substituted or unsubstituted C₁-C₁₀ alkyl group, O—, or —S—. In this regard, the C₁-C₁₀ alkyl group may include, for example, methyl, ethyl, propyl, isopropyl, butyl, and isobutyl. The substituent may include halogen. The —NR₂— may include —NH—, —N(CH₃)—, —N(CH₂CH₃)—, etc. At least one of Q and Q₁ includes at least one of primary and secondary amino groups.

In one embodiment, R₁ is selected from a group consisting of a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group. The substituent may be halogen, —COOH, —SO₃H, —SO₂H, —SOH, —H₂PO₄, —HPO₄⁻, or —PO₄²⁻. The substituted or unsubstituted C₁-C₁₀ alkyl group may be methyl, ethyl, propyl, isopropyl, butyl, or isobutyl. The substituted or unsubstituted C₃-C₁₀ cycloalkyl group may be cyclopropyl, cyclobutyl, or cyclopentyl. The substituted or unsubstituted C₂-C₁₀ alkenyl group may be ethenyl, propenyl, or butenyl. In addition, the substituted or unsubstituted C₂-C₁₀ alkynyl group may be ethynyl, propynyl, or butynyl.

In one embodiment, Y1 is a group represented by

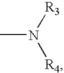

wherein R₃ and R₄ are each independently selected from a group consisting of hydrogen, a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group, or R₃ and R₄ may be bound to each other to form an alicyclic or aromatic ring. The substituent may be halogen, or a primary, secondary or tertiary amino group. The alicyclic ring may be a 3- to 8-membered cycloalkyl ring or cycloalkene ring. The aromatic ring may be a 4- to 8-membered aromatic ring.

In another embodiment, Y₁ may be —NH₂, —NH(CH₃), or —NH(CH₂CH₃).

In another embodiment, Y₁ is a group that is represented by

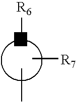

and has a 3- to 8-membered alicyclic ring or a 4- to 8-membered aromatic ring, where R⁶ and R₇ are each independently selected from a group consisting of hydrogen, halogen (with a proviso that R₆ is not halogen), a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group. The substituent may include halogen.

In still another embodiment, Y₁ may be

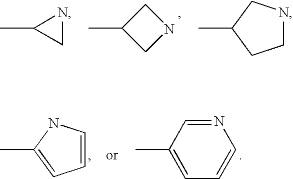

In another embodiment, in Formula 1, Q or Q₁ is a group represented by X₁—R₁—Y₁, wherein X₁ and Y₁ is as defined in the above and R₁ may be selected from a group consisting of a C₁-C₁₀ alkyl group, a C₃-C₁₀ cycloalkyl group, a C₂-C₁₀ alkenyl group, and a C₂-C₁₀ alkynyl group which is substituted with at least one selected from the group consisting of —COOH, —SO₃H, —SO₂H, —SOH, —H₂PO₄, —HPO₄⁻, and —PO₄²⁻.

In another embodiment, in Formula 1, Q or Q₁ may also be selected from a group consisting of

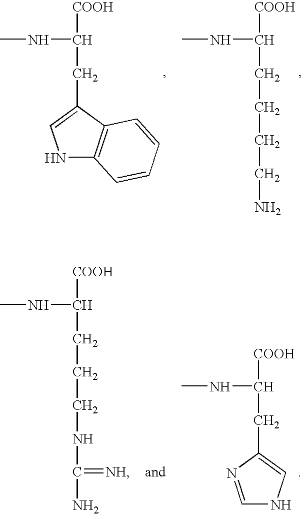

In one embodiment, the compound of Formula 1 may be prepared by reacting a dianhydride with a compound with a functional group that may react with the dianhydride. The dianhydride may be, for example,

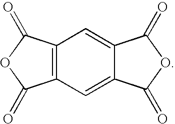

The functional group of the compound that may react with the dianhydride may be, for example, an amino group. In one embodiment the functional group may be ethylene diamine or 1-(3-aminopropyl)imidazole).

In one embodiment, the bi-functional material may be a polymer including at least one monomer selected from a group consisting of monomers represented by Formula M0, M1, M2, and M3 below, wherein the polymer includes at least one monomer having A moiety and at least one monomer having B moiety.

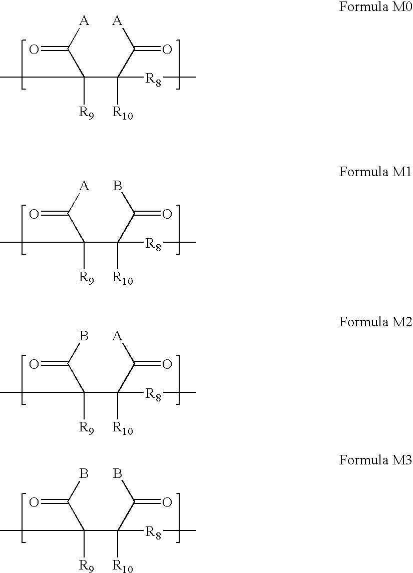

Here, A is a group represented by —OH or —X₂—R₁₁—Y₂, wherein X₂ is —NR₁₂— where R₁₂ is selected from the group consisting of hydrogen and a substituted or unsubstituted C₁-C₁₀ alkyl group, O—, or —S—, R₁₁ is selected from a group consisting of a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group, Y₂ is —COOH, —SO₃H, —SO₂H, —SOH, —H₂PO₄, —HPO₄⁻, or —PO₄², and

B is a group represented by —X₃—R₁₃—Y₃, wherein X₃ is —NR₁₄—, wherein R₁₄ is selected from a group consisting of hydrogen, a substituted or unsubstituted C₁-C₁₀ alkyl group, —O— and —S—, R₁₃ is selected from a group consisting of a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group, Y₃ is a group represented by

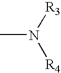

wherein R₃ and R₄ are each independently selected from a group consisting of hydrogen, a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group, or R₃ and R₄ are bound to each other to form an alicyclic or aromatic ring.

In another embodiment, Y₃ is a group that is represented by

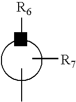

and has a 4- to 8-membered alicyclic ring or a 6- to 8-membered aromatic ring, where R₆ and R₇ are each independently selected from a group consisting of hydrogen, halogen (with a proviso that R₆ is not halogen), a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group. R₈ may be selected from a group consisting of a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group, and R₉ and R₁₀ are each independently selected from a group consisting of hydrogen, halogen, a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group. A and B may have a polymerization degree of about 2 to about 30,000.

In Formula M0, M1, M2, and M3, A is a group represented by —OH or —X₂—R₁₁—Y₂, wherein X₂ is —NR₁₂— where R₁₂ is a group selected from hydrogen and a substituted or unsubstituted C₁-C₁₀ alkyl group, O—, or —S—. Here, the C₁-C₁₀ alkyl group may be, for example, methyl, ethyl, propyl, isopropyl, butyl, or isobutyl. The substituent may include halogen. The —NR₁₂— may be —NH—, —N(CH₃)—, or —N(CH₂CH₃)—.

In one embodiment, R₁₁ may be selected from a group consisting of a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group. The substituent may be halogen, —COOH, —SO₃H, —SO₂H, —SOH, —H₂PO₄, —HPO₄⁻, or —PO₄²⁻. The substituted or unsubstituted C₁-C₁₀ alkyl may be methyl, ethyl, propyl, isopropyl, butyl, or isobutyl. The substituted or unsubstituted C₃-C₁₀ cycloalkyl group may be cyclopropyl, cyclobutyl, or cyclopentyl. The substituted or unsubstituted C₂-C₁₀ alkenyl group may be ethenyl, propenyl, or butenyl. In addition, the substituted or unsubstituted C₂-C₁₀ alkynyl group may be ethynyl, propynyl, or butynyl.

In one embodiment, Y₂ may be —COOH, —SO₃H, —SO₂H, —SOH, —H₂PO₄, —HPO₄⁻, or —PO₄²⁻.

In one embodiment, B is a group represented by —X₃—R₁₃—Y₃, wherein X₃ is —NR₁₄—, wherein R₁₄ is selected from a group consisting of hydrogen, a substituted or unsubstituted C₁-C₁₀ alkyl group, —O— and —S—. In this regard, the C₁-C₁₀ alkyl group may be methyl, ethyl, propyl, isopropyl, butyl, or isobutyl. The substituent may be halogen. The —NR₁₄— may be —NH—, —N(CH₃)—, or —N(CH₂CH₃)—.

In one embodiment, R₁₃ may be selected from a group consisting of a substituted or unsubstituted C₁-C₁₀ alkyl group, a substituted or unsubstituted C₃-C10 cycloalkyl group, a substituted or unsubstituted C₂-C₁₀ alkenyl group, and a substituted or unsubstituted C₂-C₁₀ alkynyl group. The substituent may be halogen, —COOH, —SO₃H, —SO₂H, —SOH, —H₂PO₄, —HPO₄⁻, or —PO₄²⁻. The substituted or unsubstituted C₁₀-C₁₀ alkyl group may be methyl, ethyl, propyl, isopropyl, butyl, or isobutyl. The substituted or unsubstituted C₃-C₁₀ cycloalkyl group may be cyclopropyl, cyclobutyl, or cyclopentyl. The substituted or unsubstituted C₂-C₁₀ alkenyl group may be ethenyl, propenyl, or butenyl. In addition, the substituted or unsubstituted C₂-C₁₀ alkynyl group may be ethynyl, propynyl, or butynyl.

In one embodiment, Y₃ is a group represented by

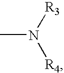

wherein R₃ and R₄ are each independently selected from a group consisting of hydrogen, a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group, or R₃ and R₄ are bound to each other to form an alicyclic or aromatic ring. The substituent may be halogen, or a primary, secondary, or tertiary amino group. The alicyclic ring may be a 3- to 8-membered cycloalkyl ring or cycloalkene ring. The aromatic ring may be a 4- to 8-membered aromatic ring. Y₃ may be —NH₂, —NH(CH₃), or —NH(CH₂CH₃).

In another embodiment, Y₃ is a group that is represented by

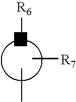

and has a 3- to 8-membered alicyclic ring or a 4- to 8-membered aromatic ring, where R⁶ and R₇ are each independently selected from a group consisting of hydrogen, halogen (with a proviso that R₆ is not halogen), a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group. The substituent may include halogen.

In still another embodiment, Y₁ may be

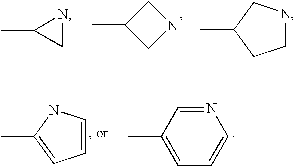

R₈ may be selected from a group consisting of a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group. The substituent may be halogen.

R₉ and R₁₀ are each independently selected from a group consisting of hydrogen, halogen, a substituted or unsubstituted C₁-C₁₀ alkyl group, a substituted or unsubstituted C₃-C₁₀ cycloalkyl group, a substituted or unsubstituted C₂-C₁₀ alkenyl group, and a substituted or unsubstituted C₂-C₁₀ alkynyl group. The substituent may be halogen.

In one embodiment, the compound including a monomer having A moiety and a monomer having B moiety may have a polymerization degree of about 2 to about 30.

In the compound including a monomer having A moiety and a monomer having B moiety, B may be a compound selected from a group consisting of a C₁-C₁₀ alkyl group, a C₃-C₁₀ cycloalkyl group, a C₂-C₁₀ alkenyl group, and a C₂-C₁₀ alkynyl group, wherein R₁₄ is substituted with at least one selected from the group consisting of —COOH, —SO₃H, —SO₂H, —SOH, —H₂PO₄, —HPO₄⁻, and —PO₄²⁻.

In the compound including a monomer having A moiety and a monomer having B moiety, B may be selected from a group consisting of

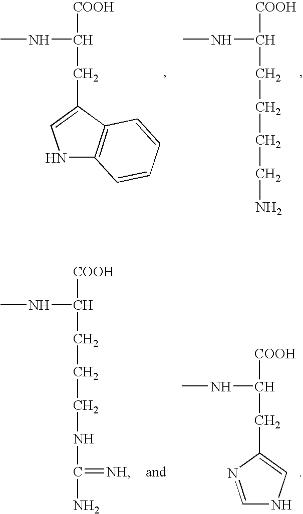

In one embodiment, the compound including a monomer having A moiety and a monomer having B moiety may be prepared by hydrolyzing a polyanhydride (for example, poly(ethylene-alt-maleic anhydride) (molecular weight: 100,000-500,000) to expose a carboxyl group, reacting the carboxyl group with a material such as, N-hydroxysuccineimide or 1,[3-(dimethylamino)propyl]-3-ethylcarbodiimide to activate the carboxyl group by an ester bond, and then coupling-reacting the activated carboxyl group with A (for example, H₂O) or B (for example, 1-(3-aminopropyl)imidazole). Alternatively, the compound including a monomer having A moiety and a monomer having B moiety may be prepared by directly reacting a polyanhydride (for example, poly(ethylene-alt-maleic anhydride) (molecular weight: 100,000-500,000) with a reactant including A (for example, H₂O) or a reactant including B (for example, 1-(3-aminopropyl)imidazole). In this regard, a net charge of the compound including a monomer having A moiety and a monomer having B moiety may be adjusted by having A or B contained in the compound at an appropriate ratio in the reaction.

In addition, the hydrophobic material is a material having a surface with a water contact angle in a range of about 70 to about 90 degrees. The surface of the hydrophobic material may be formed of octadecyltrichloro silane (“OTS”), tridecafluorotetrahydrooctyl trimethoxy silane (“DTS”), octadecyldimethyl(3-trimethoxysilylpropyl)ammonium chloride (“OTC”), polyethyleneiminetrimethoxy silane (“PEIM”), or the like.

The water contact angle is measured using a Kruss prop Shape Analysis System type DSA 10 Mk2. 1.5 μl of distilled water is automatically dropped on the sample. The water droplets are monitored using a CCD camera every 2 seconds for 10 seconds and analyzed using Drop Shape Analysis software (DSA version 1.7, Kruss). The profile of the water droplet is measured using a conic section equation. The angles are measured from right and left sides. 5 droplets per a sample are measured. The average water contact angle of 5 water droplets is used.

Referring to the solid support, the CST material is a product having a solid phase used to reversibly bind with nucleic acid in the sample, wherein the product includes a plurality of positively ionizable groups which are immobilized onto the solid phase, efficiently binds to nucleic acids in the sample at the first pH at which the positively ionizable groups are positively charged, efficiently releases the bound nucleic acids at the second pH at which the positively ionizable groups are negatively charged, neutralized, or weakly positively charged, and are supplied by chemical species selected from a group consisting of a biological buffer, polyhydroxylated amine, histidine, and polyhistidine, and have a pKa in a range of about 4.5 to about 8.5. The CST material may be a biological buffer selected from a group consisting of N-2-acetamido-2-aminoethanesulfonic acid (“ACES”); N-2-acetamido-2-iminodiacetic acid (“ADA”); N,N-bis2-hydroxyethyl-2-aminoethanesulfonic acid (“BES”); N,N-bis-2-hydroxyethylglycine (“BICINE”); bis-2-hydroxyethyliminotrishydroxymethylmethane (“Bis-Tris”); 1,3-bistrishydroxymethylmethylaminopropane (“Bis-Tris propane”); 3-N,N-bis-2-hydroxyethylamino-2-hydroxypropanesulfonic acid (“DIPSO”); 2-hydroxyethylpiperazine-N-3-propanesulfonic acid (“EPPS”); N-(2-hydroxyethyl)piperazine-N-4-butanesulfonic acid (“HEPBS”); N-(2-hydroxyethyl)piperazine-N-2-ethanesulfonic acid (“HEPES”); N-(2-hydroxyethyl)piperazine-N-2-propanesulfonic acid (“HEPPSO”); 2-N-morpholinoethanesulfonic acid (“MES”); 4-N-morpholinobutanesulfonic acid (“MOBS”); 3-N-morpholinopropanesulfonic acid (“MOPS”); 3-N-morpholino-2-hydroxypropanesulfonic acid (“MOPSO”); piperazine-N-N-bis-2-ethanesulfonic acid (“PIPES”); piperazine-N-N-bis-2-hydroxypropanesulfonic acid (“POPSO”); N-trishydroxymethyl-methyl-3-aminopropanesulfonic acid (“TAPS”); 3-N-trishydroxymethyl-methylamino-2-hydroxypropanesulfonic acid (“TAPSO”); N-trishydroxymethyl-methyl-2-aminoethanesulfonic acid (“TES”); N-trishydroxymethylmethylglycine (“TRICINE”); trishydroxymethylaminomethane (“Tris”); polyhydroxlyated imidazole; and triethaneolamine dimer, and a polymer thereof.

The solid support material may be in the form of a bead, a sphere, a plate, a pillar, a sieve, a filter, gel, a membrane, a fiber, a tube, a well, or a channel in a microfluidic device. The solid support material may be magnetic. The solid support material may be formed of glass, silica, latex, or a polymerizable material.

The separation of the solid support material from the mixture may be performed using known methods, such as centrifugation or magnetic force when the solid support is magnetic. The centrifugation may be performed at a low centrifugal force. For example, the centrifugation may be performed at a force of about 1,500 to about 6,000 g.

In one embodiment, the method may further include separating gDNA from the separated gDNA-solid support material complex. The separation of gDNA from the solid support material may be performed by eluting the gDNA from the gDNA-solid support material complex. The elution may be performed at a temperature ranging from about 25 to about 70°. For example, the elution may be performed at 65° at a pH of 8.9 in the presence of 10 mM Tris HCl.

In another embodiment, the method may further include separating gDNA from the separated solid support by contacting the gDNA with another solid support which is more specifically bound to the gDNA in a certain buffer condition to form another gDNA-solid support complex, and eluting the gDNA from the complex. For example, the separation of gDNA may be performed by contacting the solution with another solid support coated with a bi-functional material, a CST material, or a hydrophobic material, separating the bi-functional material, the CST material, or the hydrophobic material from the solution, and eluting gDNA from the separated bi-functional material, CST material, or hydrophobic material. The contact may be performed in a solution including kosmotropic salt in a concentration of about 0.3 to about 3M and chaotropic salt in a concentration of about 3 to about 6M at a pH ranging from about 3 to about 5. The bi-functional material, the CST material, or the hydrophobic material may be separated from the solution using gravity or centrifugal force. The centrifugation may be performed at about 1,500 to about 6,000 g. The elution may be performed at a temperature ranging from about 25 to about 70° C. For example, the elution may be performed at 65° at a pH of 8.9 in the presence of 10 mM Tris HCl.

The method may further include separating pDNA from the remaining mixture from which the solid support material is removed. The separation of the pDNA may be performed by contacting the remaining mixture with a solid support material capable of binding to pDNA to form a pDNA-solid support complex, and eluting pDNA from the complex. The solid support may be a metal oxide, such as alumina, a titanium oxide, or a material having SiO₂ structure (e.g., silica). The separation of the pDNA may be performed by contacting the solution with silica, separating the silica from the solution, and eluting pDNA from the separated silica. The contact may be performed in a solution including kosmotropic salt in a concentration of about 0.3 to about 3M and chaotropic salt in a concentration of about 3 to about 6M at a pH ranging from about 3 to about 5. The silica may be separated from the solution using gravity or centrifugal force. The centrifugation may be performed at about 1,500 to about 6,000 g. The elution may be performed at a temperature ranging from about 25 to about 70°. For example, the elution may be performed at 65° at pH 8.9 in the presence of 10 mM Tris HCl.

The separation of the gDNA and pDNA may include separating a supernatant from a precipitate by centrifuging the mixture. The centrifugation may be performed at about 1,500 to about 6,000 g. The precipitate may include a precipitate of cell lysates.

In one embodiment, the method may further include separating gDNA from the precipitate. The separation of gDNA from the precipitate may be performed by suspending the precipitate in a medium, contacting the suspension with a solid support binding to gDNA to form a gDNA-solid support complex, and separating gDNA from the gDNA-solid support complex. The solid support is as defined in the above.

In one embodiment, the method may further include separating pDNA from the supernatant. The separation of pDNA from the supernatant may be performed by contacting a solid support capable of binding to the pDNA with the supernatant to form a pDNA-solid support complex, and eluting pDNA from the solid support in a condition for elution of the pDNA-solid support complex (for example, by changing pH). The solid support utilized to form a pDNA-solid support complex may include a metal oxide, such as alumina, titanium oxide, or a material having SiO₂ structure (e.g., silica). For example, the separation of pDNA may be performed by contacting the supernatant with silica, separating the silica from the supernatant, and eluting pDNA from the separated silica. The contact may be performed in a solution including kosmotropic salt in a concentration of about 0.3 to about 3M and chaotropic salt in a concentration of about 3 to about 6M at a pH ranging from about 3 to about 5. The silica may be separated from the supernatant using gravity or centrifugal force. The centrifugation may be performed at about 1,500 to about 6,000 g. The elution may be performed at a temperature ranging from about 25 to about 70°. For example, the elution may be performed at 65° at pH 8.9 in the presence of 10 mM Tris HCl.

One or more embodiments include an analytical material for separating gDNA and pDNA from each other in a sample including a kosmotropic salt, a chaotropic salt, and a material that binds to DNA at a pH of about 3 to about 5. The kosmotropic salt and the chaotropic salt are as described above.

The analytical material may be a kit. The concentration of the kosmotropic salt may be equal to or greater than 0.3 M, for example, in a range of about 0.3 to about 3 M.

When the chaotropic salt is mixed with the sample, the concentration of the chaotropic salt may be equal to or greater than 3M, for example, in a range of about 3 to about 6 M.

In the analytical material, the concentration of the kosmotropic salt may be in a range of about 0.3 to about 3 M, and the concentration of the chaotropic salt may be in a range of about 3 to about 6 M.

In one embodiment, the analytical material comprises a material that binds to DNA at a pH of about 3 to about 5 selected from a group consisting of a bi-functional material, a hydrophobic material, and a CST material. The bi-functional material, the hydrophobic material, and the CST material are described above.

The analytical material may be accompanied by a manual explaining a process of separating gDNA and pDNA from each other in a sample. In the manual, the method of separating gDNA and pDNA as described above may be described.

The analytical material may further include a metal oxide, such as alumina, titanium oxide, or a material having a SiO₂ structure (e.g., silica). The metal oxide, e.g., silica, is described above. The metal oxide, e.g., silica, may be used to selectively bind gDNA to a material binding to DNA at a pH ranging from about 3 to about 5, and selectively separate pDNA remaining in a supernatant. The use of the metal oxide, e.g., silica is described above with reference to the separation of gDNA and pDNA.

Hereinafter, one or more embodiments will be described in detail with reference to the following examples. However, these examples are not intended to limit the purpose and scope of the invention.

### Example 1

### Separation of gDNA and pDNA Using High Concentration Kosmotropic Salt, Chaotropic Salt, Bi-Functional Material, and Silica

For this example, pDNA and gDNA were separated from bacteria lysates.

First, E. coli transformed with pET-21b plasmid (4.5 kb, Novagen) was cultured in a LB medium containing 50 μg/μl of ampicillin at 37° for 16 hours while shaking at 200 rpm. The culture solution (OD₆₀₀=1.0) was centrifuged at 8,000 rpm for 10 minutes to obtain E. coli pellets. Next, 250 μl of a resuspension buffer (50 mM Tris HCl, 10 mM EDTA, 100 μg/ml RNaseA; pH 8.0) was added to a tube containing the pellets to resuspend the E. coli pellets.

250 μl of a lysis buffer (200 mM NaOH 1% SDS (w/v); pH 12 to 12.5) was added to the E. coli suspension to lyse cells. Then, 100 μl of a binding buffer (0.9 M potassium acetate and 4.2 M guanidine hydrochloride; pH 3.4 to 4.5) was added to the obtained cell lysates.

Then, 0.015 g of bi-functional material beads, discussed in detail below, was added to the cell lysate mixture and the mixture was left to sit at room temperature for 3 to 5 minutes to bind DNA with the bi-functional material beads. Then, the mixture was centrifuged at 3,000 rpm for 3 minutes to separate the bi-functional material beads from a supernatant. Next, 0.015 g of silica beads (SUNSIL-130, average diameter: 15.0 μm, Sunjin Chemical Co., Ltd.) was added to the supernatant from which the bi-functional material beads were removed, and the solution was left to sit at room temperature for 3 to 5 minutes to bind DNA to the silica beads.

Finally, 100 μl of an elution buffer (10 mM Tris HCl, pH 8.5) was added to the silica beads, and the solution was left to sit at 65° for 3 to 5 minutes to elute DNA. In addition, 100 μl of an elution buffer (10 mM Tris HCl, pH 8.9) was added to the bi-functional material beads, and the solution was left to sit at 65° for 3 to 5 minutes to elute DNA. Nucleic acids in the solution were identified using electrophoresis in 0.8% agarose.

As control 1, DNA was separated using a commercially available bacteria gDNA separation kit, QIAamp DNA Mini Kit™ (Qiagen™, Catalog No. 51304) according to a manual (QIAamp DNA Mini™ and Blood Mini™ Handbook, 2^{nd} edition, November 2007).

As control 2, DNA was separated using a binding buffer having different salt concentrations, and bi-functional material beads. For example, 250 μl of a lysis buffer (200 mM NaOH 1% SDS (w/v): pH 12 to 12.5) was added to an E. coli suspension to lyse the cells. Then, 1 ml of a binding buffer (100 mM sodium acetate buffer, pH 4) was added to the obtained cell lysates. Then, 0.015 g of bi-functional material beads were added to the cell lysate mixture and the mixture was left to sit at room temperature for 3 to 5 minutes and centrifuged at 3,000 rpm for 3 minutes to separate the bi-functional material beads from the supernatant. The separated bi-functional material beads were suspended in 10 mM Tris-HCl at pH 7, and the suspension was centrifuged at 3,000 rpm for 3 minutes to wash the bi-functional material beads. The washed bi-functional material beads were suspended in 100 mM Tris-HCl buffer at pH 9, and left to sit at 65° for 3 to 5 minutes to elute DNA. Then, the resultant was centrifuged at 3,000 rpm for 3 minutes to separate the bi-functional material beads from the solution to obtain a supernatant containing DNA. Nucleic acids in the solution were identified using electrophoresis in 0.8% agarose.

For this example, the bi-functional material beads were prepared using the following process. First, poly(ethylene-alt-maleic anhydride) (average molecular weight: 100,000-500,000; n=900-4,000) were immobilized onto magnetic beads (Invitrogen Dyanl AS, Catalog No. 161-02, Dynabeads® M-270 Amine, 2×10⁹ beads/ml, diameter: 2.8 μm) coated with an amino group, and the beads were subjected to reaction with 1-(3-aminopropyl)imidazole to prepare a material having a carboxyl group and an imidazole group and positively charged at a first pH and negatively charged at a second pH. Here, the first pH was in a range of about 2 to about 5, and the second pH was in a range of about 7 to about 12.

The magnetic beads coated with the amino group were immersed in 200 mM of poly(ethylene-alt-maleic anhydride (average molecular weight 100,000-500,000, n=900-4,000) based on a repeating unit of ethylene-maleic anhydride in N-methyl-2-pyrrolidone (“NMP”) and cultured at room temperature for 1 hour. As a result, poly(ethylene-alt-maleic anhydride) was subjected to reaction with the amino group of the magnetic beads to be fixed on a substrate. After the reaction, the magnetic beads were washed with ethanol and dried.

Then, the magnetic beads to which the polyanhydride (poly(ethylene-alt-maleic anhydride) was immobilized were incubated in 400 mM 1-(3-aminopropyl)imidazole solution and 600 mM water solution in N-methyl-2-pyrrolidone (“NMP”) at room temperature for 1 hour. Then, the beads were washed with ethanol. As a result, a compound having a carboxyl group and an imidazole group and positively charged at a first pH and negatively charged at a second pH was prepared. The obtained compound was washed with ethanol and dried.

The obtained compound was a bi-functional compound bound to at least one monomer selected from a group consisting of monomers represented by Formulas M0, M1, M2, and M3 below including at least monomer having A moiety and at least monomer having B moiety.

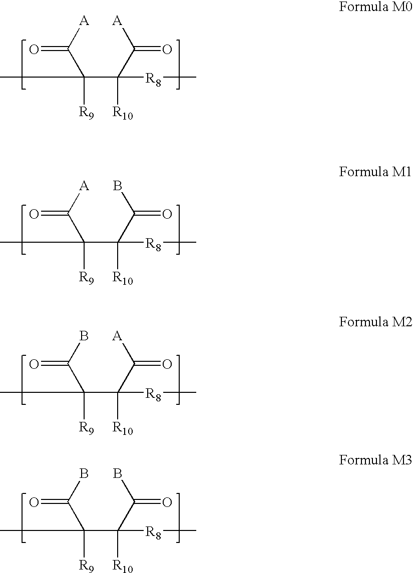

Here, A is —OH, B is

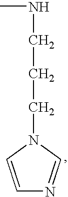

R₈ is ethyl, and R₉ and R₁₀ are hydrogen.

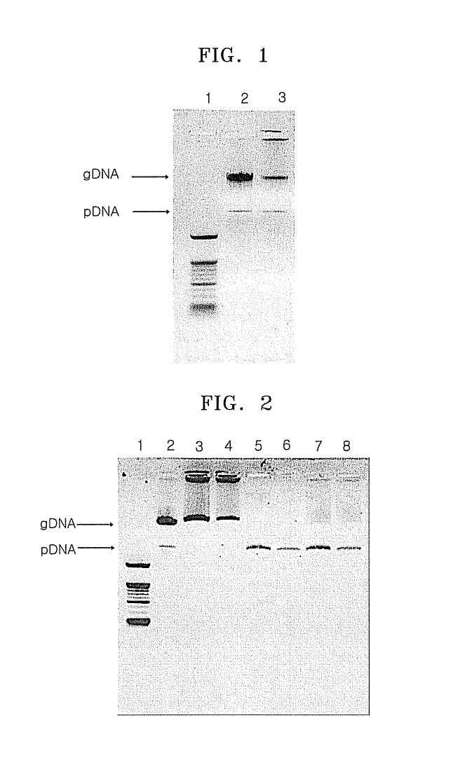

XPS analysis and TOF-SIMS analysis of the surface of the beads to which the prepared compound was immobilized were performed as described in Example 1. X-ray photoelectron spectroscopy (XPS) is a quantitative spectroscopic technique that measures the elemental composition, empirical formula, chemical state and electronic state of the elements that exist within a material. XPS spectra are obtained by irradiating a material with a beam of X-rays while simultaneously measuring the kinetic energy (KE) and number of electrons that escape from the top 1 to 10 nm of the material being analyzed. A typical XPS spectrum is a plot of the number of electrons detected (sometimes per unit time) (Y-axis, ordinate) versus the binding energy of the electrons detected (X-axis, abscissa). Each element produces a characteristic set of XPS peaks at characteristic binding energy values that directly identify each element that exist in or on the surface of the material being analyzed. Time-of-Flight Secondary Ion Mass Spectrometry (TOF-SIMS) uses a pulsed primary ion beam to desorb and ionize species from a sample surface. The resulting secondary ions are accelerated into a mass spectrometer, where they are mass analyzed by measuring their time-of-flight from the sample surface to the detector. TOF-SIMS provides spectroscopy for characterization of chemical composition, imaging for determining the distribution of chemical species, and depth profiling for thin film characterization. According to the XPS analysis, the compound contains a carbonyl group including —OH. In addition, according to the TOF-SIMS analysis, the existence of an imidazolyl group was identified. FIG. 1 is an electrophoretogram illustrating DNA separated from controls 1 and 2 according to Example 1. In FIG. 1, lane 1 illustrates results of a DNA ladder (25/100 bp mixed DNA ladder, Bioneer, Korea, Catalog No. D-1020), lane 2 illustrates results of control 1, and lane 3 illustrates results of control 2. Referring to FIG. 1, in controls 1 and 2, gDNA and pDNA were identified in the finally eluted solution using an electrophoretogram, and the amount of the gDNA and pDNA was identified with the naked eye. Thus, the efficiency of the gDNA and pDNA separations was not sufficiently high.

FIG. 2 is an electrophoretogram illustrating gDNA and pDNA separated according to Example 1, using a binding buffer containing a high concentration kosmotropic salt and chaotropic salt. In FIG. 2, lane 1 illustrates results of a DNA ladder (25/100 bp mixed DNA ladder, Bioneer, Korea, Catalog No. D-1020), lane 2 illustrates results of control 1, lanes 3 and 4 illustrate DNA eluted from the bi-functional material beads using 50 μl (lane 3) and 100 μl (lane 4) of elution buffers, and lanes 5 and 6 illustrate DNA eluted from the silica beads using 50 μl (lane 5) and 100 μl (lane 6) of elution buffers. Referring to FIG. 2, only gDNA existed in the solution eluted from the bi-functional material beads, and only pDNA existed in the solution eluted from the silica beads. Thus, gDNA and pDNA were efficiently separated using a binding buffer containing a high concentration kosmotropic salt and chaotropic salt, bi-functional material beads, and silica beads.

Based on the results, since the binding force between gDNA and the bi-functional material was greater than that between pDNA and the bi-functional material in the binding buffer containing a high concentration kosmotropic salt and chaotropic salt, gDNA was separated from pDNA in the form of a gDNA-bi-functional material complex, and then each of gDNA and pDNA was respectively separated from the bi-functional material beads and the solution from which the bi-functional material beads was removed. However, the process of the separation is not limited thereto.

### Example 2

### Separation of gDNA and pDNA Using High Concentration Kosmotropic Salt, Chaotropic Salt, Low Speed Centrifugation, and Silica Beads

For this example, gDNA and pDNA were separated from bacteria lysates. The separation was performed in the same manner as in Example 1, except that low speed centrifugation was used instead of using the bi-functional material. A detailed description of the process is as follows.

First, E. coli transformed with pET-21b plasmid (4.5 kb, Novagen) was cultured in a LB medium containing 50 μg/μ of ampicillin at 37° for 16 hours while shaking at 200 rpm. The culture solution (OD₆₀₀=1.0) was centrifuged at 8,000 rpm for 10 minutes to obtain E. coli pellets. 250 μl of a resuspension buffer (50 mM Tris HCl, 10 mM EDTA, 100 μg/ml RNaseA; pH 8.0) was added to a tube containing the pellets to resuspend the E. coli pellets.

Next, 250 μl of a lysis buffer (200 mM NaOH 1% SDS (w/v): pH 12 to 12.5) was added to the E. coli suspension to lyse cells. Then, 100 μl of a binding buffer (0.9 M potassium acetate and 4.2 M guanidine hydrochloride; pH 3.4 to 4.5) was added to the obtained cell lysates.

Then, the cell lysate mixture was centrifuged in a centrifuge at 4,000 to 5,000 rpm for 5 minutes to separate a crude precipitate of the cell lysates from a supernatant. 0.015 g of silica beads (SUNSIL-130, average diameter: 15.0 μm, Sunjin Chemical Co., Ltd.) were added to the supernatant from which the crude precipitate was removed, and the solution was left to sit at room temperature for 3 to 5 minutes to bind DNA to the silica beads.

Then, 100 μl of an elution buffer (10 mM Tris HCl, pH 8.5) was added to the silica beads, and the solution was left to sit at 65° for 3 to 5 minutes to elute DNA. Nucleic acids in the solution were identified using electrophoresis in 0.8% agarose.

As control 1, DNA was separated using a commercially available bacteria gDNA separation kit, QiAamp DNA Mini Kit™ (Qiagen™, Catalog No. 51304), according to a manual (QiAamp DNA Mini™ and Blood Mini™ Handbook, 2^{nd} edition, November 2007).

As control 2, 0.015 g of silica beads (SUNSIL-130, average diameter: 15.0 μm, Sunjin Chemical Co., Ltd.) were added to cell lysates suspended in a binding buffer and the suspension was left to sit at room temperature for 3 to 5 minutes to bind DNA to the silica beads without the centrifugation process. The elution process was performed in the same manner as in the experimental group.

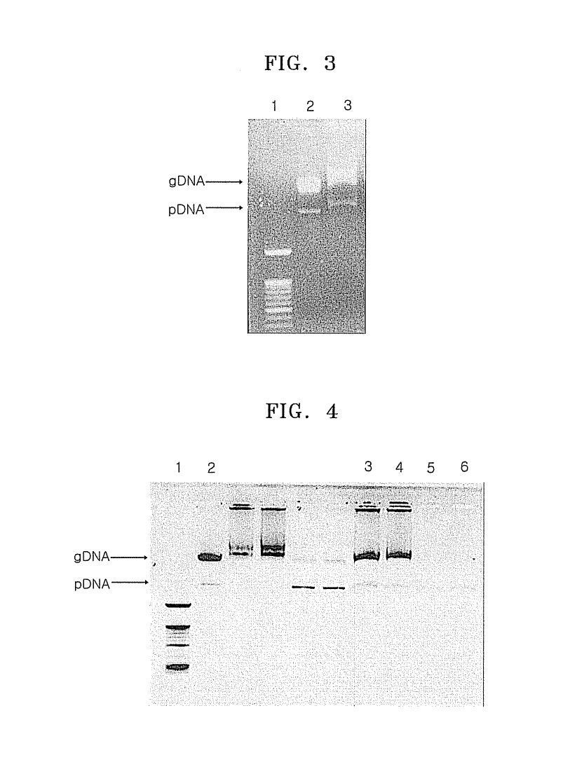

FIG. 3 is an electrophoretogram illustrating DNA separated from controls 1 and 2 according to Example 2. In FIG. 3, lane 1 illustrates results of a DNA ladder (25/100 bp mixed DNA ladder, Bioneer, Korea, Catalog No. D-1020), lane 2 illustrates results of control 1, and lane 3 illustrates results of control 2 (without centrifugation). Referring to FIG. 3, in controls 1 and 2, gDNA and pDNA were identified in the finally eluted solution using an electrophoretogram, and the amount of the gDNA and pDNA was identified with the naked eye. Thus, the efficiency of the gDNA and pDNA separations was not sufficiently high.

Lanes 7 and 8 of FIG. 2 illustrate gDNA and pDNA separated using a buffer solution containing a high concentration kosmotropic salt and chaotropic salt and low speed centrifugation, according to Example 1. Lanes 7 and 8 illustrate DNA eluted from a supernatant prepared by removing a crude precipitate by centrifuging at 4000 rpm for 5 minutes, adding silica beads to the supernatant from which the crude precipitate was removed, and eluting the DNA bound to the silica bead. Lanes 7 and 8 illustrate DNA eluted from a supernatant using 50 μl (lane 7) and 100 μl (lane 8) elution buffers, respectively. Referring to lanes 7 and 8 of FIG. 2, only pDNA existed in the solution eluted from the silica beads after the low speed centrifugation. Thus, gDNA and pDNA may be efficiently separated from each other using a binding buffer containing a high concentration kosmotropic salt and chaotropic salt, low speed centrifugation, and silica beads.

### Example 3

### Separation of gDNA and pDNA Using High Concentration Kosmotropic Salt, Chaotropic Salt, Hydrophobic Material, and Silica Beads

For this example, gDNA and pDNA were separated from bacteria lysates. The separation was performed in the same manner as in Example 1, except that a hydrophobic material was used instead of the bi-functional material. A detailed description of the process is as follows.

First, E. coli transformed with pET-21b plasmid (4.5 kb, Novagen) was cultured in a LB medium containing 50 μg/μl of ampicillin at 37° for 16 hours while shaking at 200 rpm. The culture solution (OD₆₀₀=1.0) was centrifuged at 8,000 rpm for 10 minutes to obtain E. coli pellets. 250 μl of a resuspension buffer (50 mM Tris HCl, 10 mM EDTA, 100 μg/ml RNaseA; pH 8.0) was added to a tube containing the pellets to resuspend the E. coli pellets.

Next, 250 μl of a lysis buffer (200 mM NaOH 1% SDS (w/v): pH 12 to 12.5) was added to the E. coli suspension to lyse cells. Then, 100 μl of a binding buffer (0.9 M potassium acetate and 4.2 M guanidine hydrochloride; pH 3.4 to 4.5) was added to the obtained cell lysates.

Then, 0.015 g of beads having a hydrophobic surface (silica beads SAM-coated with DTS, beads with a water contact angle: 85°, average diameter: 15 μm) were added to the mixture, and the mixture was left to sit at room temperature for 3 to 5 minutes to bind DNA to the beads having a hydrophobic surface. The mixture was centrifuged at 3,000 rpm for 3 minutes to separate the beads from a supernatant.

Then, 0.015 g of silica beads (SUNSIL-130, average diameter: 15.0 μm, Sunjin Chemical Co., Ltd.) was added to the supernatant from which the beads having a hydrophobic surface were removed, and the mixture was left to sit at room temperature for 3 to 5 minutes to bind DNA to the silica beads. Then, the mixture was centrifuged at 3,000 rpm for 3 minutes to separate the silica beads from the supernatant. Next, 100 μl of an elution buffer (10 mM Tris HCl, pH 8.5) was added to the silica beads, and the solution was left to sit at 65° for 3 to 5 minutes to elute DNA. In addition, 100 μl of an elution buffer (10 mM Tris HCl, pH 8.5) was added to the beads having a hydrophobic surface, and the solution was left to sit at 65° for 3 to 5 minutes to elute DNA. Nucleic acids in the solution were identified using electrophoresis in 0.8% agarose.

As control 1, DNA was separated using a commercially available bacteria gDNA separation kit, QIAamp DNA Mini Kit™ (Qiagen™, Catalog No. 51304), according to a manual (QIAamp DNA Mini™ and Blood Mini™ Handbook, 2^{nd} edition, November 2007).

As control 2, DNA was separated using a commercially available bacteria pDNA separation kit, QIAprep Spin MiniPrep Kit™ (Qiagen™, Catalog No. 27104), according to a manual (QIAprep Miniprep™ Handbook, 2^{nd} edition, December 2006).

The silica beads with a hydrophobic surface (average diameter 15 μm) were prepared on a solid support using a self-assembled monolayer (“SAM”) coating method according to the following process. First, silica beads were immersed in a piranha solution for more than 2 hours, washed, and dried. The tridecafluorotetrahydrooctyl trimethoxy silane (“DTS”) was mixed with toluene, and the mixture was stirred for 1 hour such that the final concentration was 100 mM. The dried silica beads were immersed in DTS solution containing toluene for 4 hours. The immersed silica beads were washed three times with EtOH for 10 minutes each, and dried. The dried silica beads were incubated at 120° for 1 hour. Thus prepared silica beads SAM-coated with DTS were used.

FIG. 4 is an electrophoretogram illustrating DNA separated using a high concentration kosmotropic salt and chaotropic salt, and a material with a hydrophobic surface according to Example 3. In FIG. 4, lane 1 illustrates results of a DNA ladder (25/100 bp mixed DNA ladder, Bioneer, Korea, Catalog No. D-1020), lane 2 illustrates results of control 1, lanes 3 and 4 illustrate DNA eluted from the magnetic beads with a hydrophobic surface using 100 μl of an elution buffer according to Example 3, and lanes 5 and 6 illustrate DNA eluted from the silica beads using 100 μl of an elution buffer according to Example 3. Referring to FIG. 4, in the solution eluted from the magnetic beads with a hydrophobic surface, gDNA mostly existed with a small minority of pDNA, and only pDNA existed in the solution eluted from the silica beads. Thus, gDNA and pDNA were efficiently separated using a binding buffer containing a high concentration kosmotropic salt and chaotropic salt, magnetic beads with a hydrophobic surface, and silica beads.

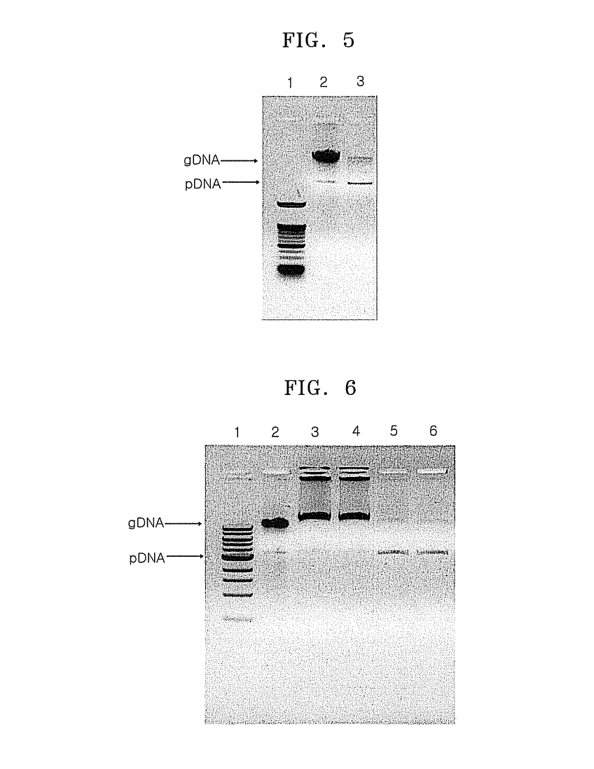

FIG. 5 is an electrophoretogram illustrating DNA separated from controls 1 and 2 of according to Example 3. In FIG. 5, lane 1 illustrates results of a DNA ladder (25/100 bp mixed DNA ladder, Bioneer, Korea, Catalog No. D-1020), lane 2 illustrates results of control 1, and lane 3 illustrates results of control 2. As shown in FIG. 5, the purity of pDNA separated using a commercially available pDNA separation kit was less than that of pDNA separated according to Example 3.

### Example 4

### Separation of gDNA and pDNA Using High Concentration Kosmotropic Salt, Chaotropic Salt, CST Material, and Silica Beads

For this example, gDNA and pDNA were separated from bacteria lysates. The separation was performed in the same manner as in Example 1, except that a CST material was used instead of the bi-functional material. A detailed description of the process is as follows.

First, E. coli transformed with pET-21b plasmid (4.5 kb, Novagen) was cultured in a LB medium containing 50 μg/μl of ampicillin at 37° for 16 hours while shaking at 200 rpm. The culture solution (OD₆₀₀=1.0) was centrifuged at 8,000 rpm for 10 minutes to obtain E. coli pellets. 250 μl of a resuspension buffer (50 mM Tris HCl, 10 mM EDTA, 100 μg/ml RNaseA; pH 8.0) was added to a tube containing the pellets to resuspend the E. coli pellets.

Next, 250 μl of a lysis buffer (200 mM NaOH 1% SDS (w/v): pH 12 to 12.5) was added to the E. coli suspension to lyse cells. Then, 100 μl of a binding buffer (0.9 M potassium acetate and 4.2 M guanidine hydrochloride; pH 3.4 to 4.5) was added to the obtained cell lysates.

Then, 20 μl of CST magnetic beads (diameter <1 μm, concentration: 25 mg/ml, storage buffer 10 mM MES, pH 5.0, 10 mM NaCl, 0.1% Tween 20) (Invitrogen, ChargeSwitch gDNA Blood Kits; Cat. No. CS11000) were added to the cell lysate mixture, and the mixture was left to sit at room temperature for 3 to 5 minutes to bind DNA to the CST beads. Then, the CST magnetic beads were separated from the supernatant using magnetic force. 0.015 g of silica beads (SUNSIL-130, average diameter: 15.0 μm, Sunjin Chemical Co., Ltd.) were added to the supernatant from which the magnetic CST beads were removed, and the solution was left to sit at room temperature for 3 to 5 minutes to bind DNA to the silica beads.

After the silica beads were separated from the supernatant by centrifuging at 3,000 rpm for 3 minutes, 100 μl of an elution buffer (10 mM Tris HCl, pH 8.5) was added to the silica beads, and the solution was left to sit at 65° for 3 to 5 minutes to elute DNA. Furthermore, 100 μl of an elution buffer (10 mM Tris HCl, pH 8.5) was added to the CST magnetic beads, and the solution was left to sit at 65° for 3 to 5 minutes to elute DNA. Nucleic acids in the solution were identified using electrophoresis in 0.8% agarose.

FIG. 6 is an electrophoretogram illustrating DNA separated using a high concentration kosmotropic salt and chaotropic salt, and a CST material according to Example 4. In FIG. 6, lane 1 illustrates results of a DNA ladder (1 kb DNA ladder, NEB, Catalog No. N-3232S), lane 2 illustrates results of gDNA and pDNA controls (gDNA and pDNA separated from control 1 according to Example 3), lanes 3 and 4 illustrate DNA eluted from the CST beads using 100 μl of an elution buffer according to Example 4, and lanes 5 and 6 illustrate DNA eluted from the silica beads using 100 μl of an elution buffer according to Example 4. Referring to FIG. 6, only gDNA existed in the solution eluted from the CST beads, and only pDNA existed in the solution eluted from the silica beads. Thus, gDNA and pDNA were efficiently separated using a binding buffer containing a high concentration kosmotropic salt and chaotropic salt, the CST beads, and silica beads.

### Example 5

### Influence of Kosmotropic Salt on the Separation of gDNA and pDNA

In order to identify the influence of types of kosmotropic salts on the separation of gDNA and pDNA, the separation was performed in the same manner as in Example 1, except that 0.9 M sodium acetate and 4.2 M guanidine hydrochloride were used as the binding buffer instead of 0.9 M potassium acetate and 4.2 M guanidine hydrochloride.

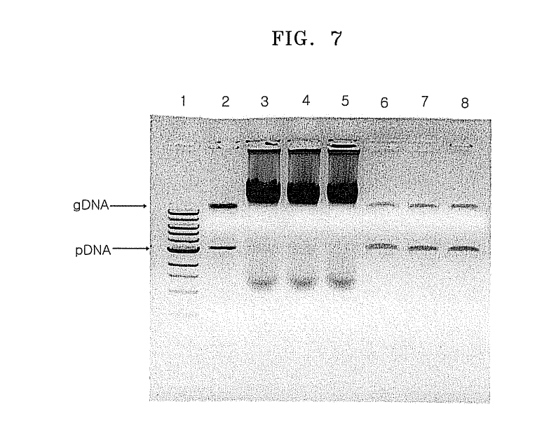

FIG. 7 is an electrophoretogram illustrating the influence of types of kosmotropic salts on the separation of gDNA and pDNA. In FIG. 7, lane 1 illustrates results of a DNA ladder (1 kb DNA ladder, NEB, Catalog No. N-3232S), lane 2 illustrates results of gDNA and pDNA controls (gDNA and pDNA separated from control 1 according to Example 3), lanes 3 and 4 illustrate DNA eluted from the bi-functional material beads using 100 μl of an elution buffer when 0.9 M potassium acetate and 4.2 M guanidine hydrochloride were used as a binding buffer, lane 5 illustrates DNA eluted from the bi-functional material beads using 100 μl of an elution buffer when 0.9 M sodium acetate and 4.2 M guanidine hydrochloride were used as the binding buffer, lanes 6 and 7 illustrate DNA eluted from the silica beads using 100 μl of an elution buffer when 0.9 M potassium acetate and 4.2 M guanidine hydrochloride were used as the binding buffer, lane 8 illustrates DNA eluted from the silica beads using 100 μl of an elution buffer when 0.9 M sodium acetate and 4.2 M guanidine hydrochloride were used as the binding buffer.

As shown in FIG. 7, the results using potassium acetate are similar to those using sodium acetate.

As described above, according to the one or more of the above embodiments, gDNA and pDNA may be efficiently separated from each other using a buffer containing a kosmotropic salt and chaotropic salt, and without using high-speed centrifugation.

According to one or more of the above embodiments, gDNA and pDNA may be efficiently separated from each other using a kit for separating gDNA and pDNA using a buffer containing a kosmotropic salt and chaotropic salt, and without using high-speed centrifugation.

While one or more embodiments of the present invention have been particularly shown and described it will be understood by those of ordinary skill in the art that various modifications in form and detail may be made therein without departing from the spirit and scope of the teachings of the present invention as defined by the following claims. The exemplary embodiments should be considered in a descriptive sense only and not for purposes of limitation. Therefore, all such modifications are intended to be included within the scope of the claims.

What is claimed is:

1. A method of separating genomic DNA and plasmid DNA from each other, the method comprising:
mixing a sample comprising genome DNA and plasmid DNA with a solution comprising a kosmotropic salt and a chaotropic salt to prepare a mixture having a pH ranging from about 3 to about 5; and
separating the genome DNA and plasmid DNA from the mixture.

2. The method of claim 1, wherein the sample comprises a lysed biological sample.

3. The method of claim 1, wherein the concentration of the kosmotropic salt is in a range of about 0.3 to about 3 M, and the concentration of the chaotropic salt is in a range of about 3 to about 6 M.

4. The method of claim 1, wherein the kosmotropic salt comprises one anion selected from a group consisting of acetate, phosphate, sulfate, and citrate, and a counter cation of the anion.

5. The method of claim 1, wherein the chaotropic salt comprises one anion selected from a group consisting of Br⁻, NO₃⁻, I⁻, SCN⁻, and ClO₄⁻, and a counter cation of the anion.

6. The method of claim 1, wherein a molar concentration ratio of the kosmotropic salt to the chaotropic salt is in a range of about 1:14 to about 1:2.

7. The method of claim 1, wherein the separating the genome DNA and plasmid DNA from the mixture comprises:
contacting the mixture with a solid support capable of binding to genome DNA and separating the solid support from the mixture; and
separating the plasmid DNA from the remaining mixture from which the solid support is separated.

8. The method of claim 7, wherein the solid support material comprises one material selected from a group consisting of a bi-functional material, a hydrophobic material, and a CST material,
wherein the bi-functional material is positively charged and bound to nucleic acids at a first pH ranging from about 2 to about 5 and negatively charged and releases the bound nucleic acids at a second pH ranging from about 7 to about 12,
wherein the hydrophobic material comprises a material having a water contact angle of about 70° to about 90°, and
wherein the CST material comprises a product having a solid phase used to reversibly bind with nucleic acid in the sample, wherein the product comprises a plurality of positively ionizable groups which are immobilized onto the solid phase, efficiently bind to nucleic acids in the sample at the first pH at which the positively ionizable groups are positively charged, efficiently releases the bound nucleic acids at the second pH at which the positively ionizable groups are negatively charged, neutralized, or weakly positively charged, are supplied by chemical species selected from a group consisting of a biological buffer, polyhydroxylated amine, histidine, and polyhistidine, and have a pKa in a range of about 4.5 to about 8.5.

9. The method of claim 7, further comprising separating the genome DNA from the separated solid support material.

10. The method of claim 7, wherein the separating the plasmid DNA from the remaining mixture is performed by
contacting the remaining mixture with a metal oxide,
separating the metal oxide from the remaining mixture, and
eluting plasmid DNA from the separated metal oxide.

11. The method of claim 1, wherein the separating the genome DNA and plasmid DNA from the mixture comprises:
separating a precipitate from a supernatant by centrifuging the mixture; and
separating the plasmid from supernatant.

12. The method of claim 11, wherein the centrifuging is performed at a force of about 1,500 to about 6,000 g.

13. The method of claim 11, further comprising separating the genome DNA from the precipitate.

14. The method of claim 11, wherein the separating the plasmid DNA is performed by
contacting the supernatant with a metal oxide,
separating the metal oxide from the supernatant, and
eluting plasmid DNA from the separated metal oxide.

15. The method of claim 10, wherein separating the metal oxide from the remaining mixture comprises centrifuging the mixture; and removing the supernatant.

16. The method of claim 14, wherein separating the metal oxide from the remaining mixture comprises centrifuging the mixture; and removing the supernatant.

17. An analytical material for separating genome DNA and plasmid DNA from each other in a sample comprising
a kosmotropic salt,
a chaotropic salt, and
a material that binds to DNA at a pH of about 3 to about 5.

18. The analytical material of claim 17, wherein the concentration of the kosmotropic salt is in a range of about 0.3 to about 3 M, and the concentration of the chaotropic salt is in a range of about 3 to about 6 M.

19. The analytical material of claim 17, wherein the material is one selected from a group consisting of a bi-functional material, a hydrophobic material and a CST material, wherein the bi-functional material is positively charged and bound to nucleic acids at a first pH ranging from about 2 to about 5 and negatively charged and releases the bound nucleic acids at a second pH ranging from about 7 to about 12,
wherein the hydrophobic material has a water contact angle of about 70° to about 90°, and
wherein the CST material comprises a product having a solid phase used to reversibly bind with nucleic acid in the sample, wherein the product comprises a plurality of positively ionizable groups which are immobilized onto the solid phase, efficiently bind to nucleic acids in the sample at the first pH at which the positively ionizable groups are positively charged, efficiently release the bound nucleic acids at the second pH at which the positively ionizable groups are negatively charged, neutralized, or weakly positively charged, are supplied by chemical species selected from a group consisting of a biological buffer, polyhydroxylated amine, histidine, and polyhistidine, and have a pKa in a range of about 4.5 to about 8.5.

20. The analytical material of claim 17, further comprising a manual for separating the genome DNA and plasmid DNA from each other in the sample.

21. The analytical material of claim 17, further comprising a silica material.

22. A method of separating genomic DNA and plasmid DNA from each other, the method comprising:
mixing a sample comprising genome DNA and plasmid DNA with a solution comprising a kosmotropic salt and a chaotropic salt to prepare a mixture having a pH ranging from about 3 to about 5;
contacting the mixture with a solid support material capable of binding to genome DNA to form a genome DNA-solid support material complex;
separating the genome DNA-solid support material complex from the mixture; and
separating the plasmid DNA from the remaining mixture from which the genome DNA-solid support material complex is separated,
wherein the separating the plasmid DNA from the remaining mixture is performed by
contacting the remaining mixture with a metal oxide,
separating the metal oxide from the remaining mixture, and
eluting plasmid DNA from the separated metal oxide.
